(19) United States
(12) Patent Application Publication
(10) Pub. No.: US 2010/0041076 A1
(43) Pub. Date: Feb. 18, 2010

(54) Reagents, Methods and Kits for the Universal Rapid Immuno-Detection

(75) Inventors: Ran Zou, Hillsborough, NJ (US); Song Zou, Fuzhou (CN)

Correspondence Address: GRAHAM CURTIN, P.A., 4 Headquarters Plaza, P.O. Box 1991, Morristown, NJ, 07962-1991, US

(73) Assignee: EZ BIO INC., Hillsborough, NJ (US)

(21) Appl. No.: 12/190,263

(22) Filed: Aug. 12, 2008

Related U.S. Application Data
(63) Continuation of application No. 11/089,059, filed on Mar. 24, 2005, now abandoned.
(60) Provisional application No. 60/556,065, filed on Mar. 25, 2004.

(51) Int. Cl. G01N 33/53; G01N 33/543

(52) U.S. Cl. 435/7.92; 436/518; 435/7.1

(57) ABSTRACT

This invention relates to a novel immuno-detection methods, kits and reagents. The Combination of this invention, combining at least two of the following reagents of a Non-specific Competitor, a Specific Indicator, a primary antibody and an antigen, provides a faster and easier method for an immuno-detection, combining at least two of the following steps of blocking, antigen binding, primary antibody binding and 2^{nd} antibody binding in an immuno-detection into a one step reaction. The significant specificity of this invention is to combine blocking, primary antibody binding and 2^{nd} antibody binding in an immuno-detection into a one step reaction. The immuno-detection process of this invention includes 3 steps: 1) one-step rapid reaction; 2) washing; and 3) developing. The whole process takes as short time as 30 minutes.

### CROSS REFERENCE TO RELATED APPLICATIONS

This application claims the benefit of the filling date of U.S. Provisional Patent Application No. 60/556,065, filed Mar. 25, 2004, the contents of which is incorporated by reference in its entirety.

### FIELD OF THE INVENTION

The present invention relates to reagents, methods and kits for rapidly detecting proteins in the field of immuno-detection.

### BACKGROUND OF THE INVENTION

Immuno-detection or immunoassay is a powerful and highly sensitive method for detection of specific proteins. Two most typical immunoassays, Enzyme-Linked Immunosorbent Assay (ELISA) and Western blot-detection (WB) were developed in 1970s. With the success of development of horseradish peroxidase (HRP)-conjugated antibody (1974, Nakane and Kawaoi) and the development of alkaline phosphatase (AP)-conjugated antibody (Voller at el), immunoassay has become a vital tool and widely employed in identification or quantification for specific proteins.

A regular procedure of immuno-detection, such as Western blot, normally includes 7 steps: 1) blocking; 2) washing; 3) binding of primary antibody; 4) washing; 5) binding of conjugated probe antibody (2^{nd} antibody); 6) further washing; and 7) developing (calorimetric/autoradiographic/chemiluminescent detection). The whole process is time-consuming and labor-intensive, usually taking about 5 to 7 hours. There is a significant need for a convenient, relative easy and fast immunoassay.

Many improvements for immunoassays have been developed. These improvement has mainly been either one or a combination of the following: 1) Using pre-coated and blocked solid phase (assay plates or strips) (such as: Osther, et al., U.S. Pat. No. 4,885,235; Osther, et al., U.S. Pat. No. 5,093,230; Urnovitz's U.S. Pat. No. 5,447,837; Rails, et al., U.S. Pat. No. 6,015,681); 2) Using pre-labeled primary antibodies (such as: Pegg, et al., U.S. Pat. No. 5,212,065; Rails, et al., U.S. Pat. No. 6,015,681; Rails, et al.'s U.S. Pat. No. 6,599,691; Stewart's U.S. Pat. No. 6,503,702; Slack, et al., WO 03/052379; Cullum, et al., WO 2005/003376); and 3) Using special or additional device (such as: Pegg, et al., U.S. Pat. No. 5,212,065; Slack, et al., WO 03/052379; Tung, et al., U.S. Pat. No. 6,627,459; Rails, et al., WO 97/05486). However, the pre-labeled primary antibody method decreases a detection sensitivity and flexibility in the choice of a primary antibody label. And pre-labeled primary antibodies are usually expensive and only available for very limited antibodies. The biggest limitation of these improved methods is that they are not universal, only suitable for detecting one or a group of specific antigens or antibodies. Furthermore, the most time- and labor-consuming steps: blocking, binding of primary antibody and binding of conjugated probe antibody (2^{nd} antibody), are still required in most of these immunoassays.

This invention describes a Universal Rapid Immuno-detection method, which combines at least two of the following steps of blocking, antigen binding, primary antibody binding and 2^{nd} antibody binding in the immuno-detection into a one-step rapid reaction (or one-step reaction). The significance of this invention is to combine steps of blocking, primary antibody binding and 2^{nd} antibody into a one-step reaction. The immuno-detection process according to this invention includes 3 steps: 1) one-step rapid reaction; 2) washing; and 3) developing. The whole process of this invention takes less than one hour.

### SUMMARY OF THE INVENTION

This invention relates to methods, kits, reagents and uses of the reagents for detecting proteins in an immuno-detection.

The advantages of the Universal Rapid Immuno-detection Method are: 1) Rapid, requiring only 0.5-1 hr (comparing that a regular immuno-assay requires 5-7 hrs); 2) Easy, using a one-step reaction instead of a multiple step procedure; 3) Adaptable, Good for automatic assays as well as manual assays; 4) Sensitive, maintaining a similar sensitivity as to other regular methods; 5) Cost-efficient, antibodies and reagents in this assay system are stable and re-useable; 6) Simple, a straight-forward process (no need to label primary antibodies, no need of an additional instrument and expertise, and no need of a complicated handling process); 7) Universal, suitable for most primary antibodies and suitable for different type of immuno-detections, such as antibody-antigen reaction based conventional immunoassays (WB, ELISA and immunohistochemistry (IHC)), as well as other assays with similar mechanisms as an antibody-antigen reaction (e.g. a receptor-ligand assay). The present invention provides a novel technology to produce rapid immuno-detection kits for WB, ELISA, IHC/immunocytochemistry (ICC) and other immunoassays, and to improve current existing methods.

In one aspect, this invention relates to a method of use of a Non-specific Competitor, which is not recognized by a primary antibody and a 2^{nd} antibody or by a receptor and a ligand used in an immuno-detection system, in an immuno-detection without a need of pre-blocking which is a separated blocking step. In another aspect, this invention relates to the method of use of the Non-specific Competitor Solution wherein the Non-specific Competitor is at a high concentration.

In one aspect, this invention relates to a Specific Indicator Solution comprising a Specific Indicator which is a pre-labeled (pre-conjugated) protein or a pre-labeled (pre-conjugated) antibody capable of specifically recognizing a primary antibody or a receptor protein without interfering with the primary antibody's antigen-binding or the receptor's ligand-binding capacity.

In another aspect, this invention relates to the Specific Indicator Solution wherein the protein or antibody of the Specific Indicator specifically recognizes a primary antibody's or receptor's any portion (a native or an artificial portion) except its antigen-binding site or ligand-binding site.

In another aspect, this invention relates to the Specific Indicator Solution wherein the artificial portion is a fusion tag. In another aspect, this invention relates the Specific Indicator Solution wherein the protein or antibody of the Specific Indicator specifically recognizes a Fc portion of a immunoglobulin or a Fc-fusion protein.

In another aspect, this invention relates to the Specific Indicator Solution wherein the protein or antibody of the Specific Indicator is selected from Protein L, Protein A, protein G, Protein A/G, Fc receptor proteins.

In another aspect, this invention relates to the Specific Indicator Solution wherein the label is a directly or indirectly detectable protein or molecule, such as colloidal gold, peroxidase, alkaline phosphatase, beta-galactosidase, beta-aminase, rhodamine, biotine, avidin, luminase, fluorescent markers, radioisotope and a mixture thereof.

The Specific Indicator Solution wherein a salt is added to the Specific Indicator Solution at a high concentration is also part of this invention.

In one aspect, this invention relates to a method of use of the Specific Indicator Solution in an immuno-detection. In another aspect, this invention relates to a method of use of the Fc receptor proteins in an immuno-detection.

This invention relates to a Combination comprising at least two of the following:

a. a Non-specific Competitor which is not recognized by a primary antibody and a 2^{nd} antibody or by a receptor and a ligand used in an immuno-detection system;

b. a Specific Indicator which is a pre-labeled (pre-conjugated) protein or a pre-labeled (pre-conjugated) antibody capable of specifically recognizing a primary antibody or a receptor protein without interfering with a primary antibody's antigen-binding or a receptor's ligand-binding capacity;

c. a primary antibody (1^{st} Ab);

d. an antigen (Ag);

and optionally, further comprising a Cofactor or a Protease Inhibitor.

In one aspect, this invention relates to the Combination wherein the Non-specific Competitor is a non-specific protein selected from normal Igs or serum from non-immunized animals which are the same species used for generation of a 2^{nd} antibody used in an immuno-detection system, albumin, casein, gelatin, chicken egg white, non-fat milk powder and a mixture thereof.

In another aspect, this invention relates to the Combination wherein a weight ratio of the non-specific competitor protein vs. antibodies used in an immuno-detection is high.

In another aspect, this invention relates to a solution of the Combination wherein a total added salt concentration is high.

In one aspect, this invention relates to the Combination comprising a Non-specific Competitor and a Specific Indicator, which can further comprising a primary antibody and further comprising an antigen.

In one aspect, this invention relates to the Combination comprising a Non-specific Competitor and a primary antibody. In another aspect, this invention relates to the Combination comprising a Specific Indicator and a primary antibody. In another aspect, this invention relates to the Combination comprising a Non-specific Competitor and an antigen.

A method of use of the Combinations according to this invention in an immuno-detection is also part of this invention.

This invention also relates to a Universal Rapid Immuno-detection method combining at least two of the steps of

a. blocking;

b. antigen binding;

c. primary antibody binding;

d. 2^{nd} antibody binding;

in an immuno-detection into a one-step reaction.

In one aspect, this invention relates to the Universal Rapid Immuno-detection method which combines blocking and primary antibody binding in an immuno-detection into a one-step reaction.

In another aspect, this invention relates to the Universal Rapid Immuno-detection method which combines blocking and 2^{nd} antibody binding in an immuno-detection into a one-step reaction.

In another aspect, this invention relates to the Universal Rapid Immuno-detection method which combines primary antibody binding and 2^{nd} antibody binding in an immuno-detection into a one-step reaction. In another aspect, this invention relates to the Universal Rapid Immuno-detection method which combines blocking, primary antibody binding and 2^{nd} antibody binding in an immuno-detection into a one-step reaction. In another aspect, this invention relates to the Universal Rapid Immuno-detection method which combines blocking, antigen binding, primary antibody binding and 2^{nd} antibody binding in an immuno-detection into a one-step reaction.

The Universal Rapid Immuno-detection method according to this invention comprises the steps of:
a) one-step rapid reaction;
b) washing;
c) developing.

In one aspect, this invention relates to a Universal Rapid Immuno-detection method wherein the Immuno-detection is selected from Immuno-blot, ELISA, Immunohitochemistry, immunocytochemistry and receptor-ligand binding assay.

A kit having component parts capable of being assembled comprising the Combinations according to this invention is also part of this invention.

This invention also relates to a Rapid Wash Solution containing a high concentration of a salt.

Other aspects of the present inventions will become apparent from the following detailed description. It should be understood, however, that the detailed description and the specific examples, while indicating preferred embodiments of the invention, are given by way of illustration only, since various changes and modification within the spirit and scope of the invention will become apparent to those skilled in the art from this detailed description. One of the examples is a receptor-ligand assay: all reagents, methods, kits and uses of the reagents can be modified within the spirit and scope of this invention apparent to those skilled in the art following the antigen-antibody case, and they are part of this invention under equivalence even they are not explicitly and specifically described or claimed.

### BRIEF DESCRIPTION OF THE DRAWINGS

FIG. 1 is a scanned reproduction of Dot-blot detection results in the examination of the blocking capacity of different Non-specific Competitors in a Rapid Dot Blot detection. Six Non-specific Competitors (BSA, non-fat milk powder, egg white, goat IgG, goat serum and gelatin) were examined in an example of Dot-blot-testing rabbit IgG using goat antibody against rabbit IgG. The results indicated that without using a Non-specific Competitor (a negative control, A), the blot was very dark and not detectable due to a high non-specific binding; in contrast, by using Non-specific Competitors (C-J), the non-specific background binding was dramatically reduced to a similar level as pre-blocked blot (B.). This result demonstrated that the combination of blocking and antibody binding into one-step for an immuno-detection using Non-specific Competitor solutions achieved the same result comparing to the regular procedure.

FIG. 2 is a scanned reproduction of Dot-blot detection results in examination of the possibility to combine two steps, primary antibody binding and 2^{nd} antibody binding, into a one-step reaction in an immuno-detection. In this experiment, testing antigens were dot-blotted onto NC membrane and pre-blocked, and then detections were carried out by two different methods, a combination of primary antibody binding and 2^{nd} antibody binding, and separated primary antibody binding and 2^{nd} antibody binding (a regular method), respectively. This result demonstrated that the combination (Panel 1) of primary antibody binding and 2^{nd} antibody binding into one-step for an immuno-detection using Specific indicator, Fc-specific 2^{nd} antibody, achieved the same result comparing to the regular procedure (Panel 2).

FIG. 3 is a graph showing comparison curves of Rapid ELISA using different types of 2^{nd} antibodies. In this experiment two different types of 2^{nd} antibodies, goat anti-mouse whole IgG-HRP (this is a very normal 2^{nd} antibody for a regular ELIA) and goat anti-mouse IgG Fc-HRP, were examined in the detection of pre-coated beta-galactosidase (antigen) by Rapid ELISA (Universal Rapid immunoassay). The result indicated that the Fc-specific 2^{nd} antibody (curve with square) worked well in the rapid indirect ELISA, while a little signal was detected in the assay using normal 2^{nd} antibody (curve with triangle), and that without primary antibody, the Fc-specific 2^{nd} antibody alone (curve with rhombus) did not show any signal. This suggested that: 1) the normal 2^{nd} antibody interferes with primary antibody's binding capacity, therefore the normal 2^{nd} antibody (anti-whole IgG) is not suitable for the Universal Rapid immunoassays, even though it is suitable for a separated procedure in a regular ELISA, and 2) the Fc-specific 2^{nd} antibody specifically bind to primary antibody without interfering with primary antibody's binding capacity, and does not non-specifically bind to antigen or other proteins, therefore the Fc-specific 2^{nd} antibody is suitable to be a Specific Indicator in a combination reaction, combining primary antibody binding and 2^{nd} antibody binding into a one-step reaction. On the figure, each indicated point with a standard deviation in the curves represents an average OD450 of two individual assays.

FIG. 4 is a scanned reproduction of Western-blot results showing similar detection sensitivities by either Universal Rapid immuno-detection and a regular immuno-detection. This experiment examined a rapid method and a regular method in an example of Western blot-detecting GST protein. The results by both methods are very similar but the testing time and procedure are great different: 30 minutes and 3 steps (by a rapid method) vs. 5 hours and 7 steps (by a regular method).

FIG. 5 is a graph showing comparison curves of Rapid immuno-detection and regular immuno-detection in indirect ELISA. This experiment examined the Universal Rapid method and a regular method in the test of GST protein by indirect ELISA. The results by both methods showed comparable sensitivity for the Universal Rapid method and the regular method. However, by using the Universal Rapid method, the testing time is much shorter (30 minutes vs. 5 hours) and the assay procedure is much simpler (3 steps vs.7 steps). On the figure, each indicated point with a standard deviation in the curves represents an average OD450 of two individual assays.

FIG. 6 is a graph showing a standard curve of the Universal Rapid immuno-detection for Capture ELISA (Sandwich ELISA). The experiment demonstrated that the Universal Rapid Immuno-detection method is also suitable for a more complicate ELISA, Capture ELISA as well as for indirect ELISA. The rapid method combined blocking, antigen binding, primary antibody binding, 2^{nd} antibody binding into a one-step rapid reaction without using pre-labeled primary antibody (most regular methods for Capture ELISA use pre-labeled primary antibody). Therefore the Universal Rapid immuno-detection method provides more flexibility for Capture ELISA, since the Rapid Capture ELISA does not depend on the availability of pre-labeled primary antibody. On the figure, each indicated point with a standard deviation in the curves represents an average OD450 of two individual assays.

FIG. 7 is a graph showing a standard curve of the Universal Rapid immuno-detection for examining the binding capacity of TNF-alpha receptor. The experiment examined TNF-alpha receptor's capacity of ligand-binding at a series concentration using the Universal Rapid Immuno-detection method. The results demonstrated that the Universal Rapid Immuno-detection method is suitable for detecting receptor's ligand-binding capacity. The rapid method successfully combined blocking, receptor-ligand binding, detector antibody binding into a one-step reaction. This experiment is also an example of using a protein with artificial portion (in this case the fusion Fe is a artificial portion) in the one-step rapid reaction system. As showed in the figure, each indicated point with a standard deviation in the curves represents an average OD450 of two individual assays.

FIG. 8 is a scanned reproduction of micro-photography showing similar results from the Universal Rapid immuno-detection and a regular immuno-detection in immunocytochemistry (ICC). This experiment examined the Universal Rapid method and a regular method in testing the expression of a Flag-tagged protein in CHO cells by immunocytochemistry (ICC). The results by both methods are very similar but the testing time and procedure are great different: 30 minutes and 3 steps (by a rapid method) vs. 5 hours and 7 steps (by a regular method). As shown in the picture, A represents the case of the regular immuno-detection, B represents the case of the rapid immuno-detection, C represents the case without immuno-detection, and the antibody-detected specific proteins on cell surface are indicated by arrows.

### DETAILED DESCRIPTION OF THE INVENTION

Methods, kits, reagents and uses of the reagents for the detection of proteins in a sample are described herein.

Using a one-step reaction to replace the time- and labor-consuming four steps procedure: blocking, primary antibody binding, washing and 2^{nd} antibody binding (which are required steps in current regular immunoassays) is the main idea and a critical key development of the present invention. In general, it is difficult to combine the four steps: blocking, primary antibody binding, washing and 2^{nd} antibody binding, into a one-step reaction, because pre-binding of 2^{nd} antibody to a primary antibody will interfere with or even inhibit the primary antibody to recognize its target (detecting antigen or protein). In addition, without pre-blocking, an antibody will non-specifically bind to the membrane or well surface where target is presenting. This non-specifically binding will cause strong non-specific background that will affect assay accuracy. For the same reason, it is difficult to combine any of the two steps of blocking, primary antibody binding and 2^{nd} antibody binding into a one-step reaction in a regular immuno-detection.

To overcome these problems, this invention has developed the Non-specific Competitor Solution, the Specific Indicator Solution and the One-step Rapid Reaction. The One-step Reaction Solution includes 1) a Non-specific Competitor; and 2) a Specific Indicator; optional 3) a Specific cofactor; and optional 4) a Protease inhibitor. The use of these reagents has lead to a novel immuno-detection which combines at least any two of the steps of blocking, primary antibody binding and 2^{nd} antibody binding into to a one step reaction, as well as combines all the three steps of blocking, primary antibody binding and 2^{nd} antibody binding into a one step reaction. In some cases, e.g., Capture ELISA, this invention is to combine at least any two of the steps of blocking, antigen binding to capture antibody (similarly, receptor binding to ligand), primary antibody binding and 2^{nd} antibody binding, as well as combines all the steps of blocking, antigen binding to capture antibody (similarly, receptor binding to ligand), washing, primary antibody binding, washing and 2^{nd} antibody binding into a one-step reaction. These immuno-detection method is good for different types of immuno-detections, not only conventional immunoassays based on an antibody-antigen mechanism such as WB, ELISA and IHC, but also other assays such as assays based on the mechanism similar to an antibody-antigen reaction such as a receptor-ligand assay, and a combination of receptor-ligand binding and antibody-antigen binding assays.

### Non-specific Competitor and Non-specific Competitor Solution

The Non-specific Competitor is a non-specific protein which is not recognized by a primary antibody and a 2^{nd} antibody, or by a receptor and a ligand, used in an immunoassay or a similar assay. The non-specific protein reduces a non-specific background by competing antibodies' non-specific binding to a testing solid phase such as, membrane or well surface instead of by pre-blocking. The use of a Non-specific competitor eliminates the need of pre-blocking.

Examples of non-specific proteins are non-immunized normal animal's IgG, serum, albumin, casein, gelatin, chicken egg white or non-fat milk powder. IgG and serum should come from the same animal species that generate 2^{nd} antibodies. IgG can be a whole segment or a fragment. Examples of albumin are the ovalbumin(OVA), or the Bovine Serum Albumin (BSA).

Also, the non-specific protein can be a single type of non-specific protein, as well as a mixture of different types of non-specific proteins.

In one embodiment, the Non-specific Competitor Solution is a protein solution containing a high concentration of the non-specific protein and the weight ratio of the non-specific protein vs. antibodies is around 1000-100,000:1. The preferable ratio is around 5,000-50,000:1.

The Non-specific Competitor Solution is prepared in a solution that optimizes antigen-antibody binding kinetics. An appropriate solution is an aqueous solution or buffer. The solution is preferably provided under conditions that will promote specific binding, minimize non-specific binding, solubilize the protein, stabilize and preserve reagent reactivity, and may contain buffers, detergents, solvents, salts, chelators, proteins, polymers, carbohydrates, sugars, and other substances known to those skilled in the art.

The buffer may be selected in accordance with the substance to be analyzed. As such a buffer, one having an appropriate ion concentration and pH which does not inactivate the substance to be analyzed and does not inhibit the antigen-antibody reaction may be used. For example, a phosphate buffer or a Tris buffer may be used. The pH of the buffer is around 3 to 10, preferably around 6 to 8.

The use of the Non-specific Competitor has made it possible to combine blocking with any following steps: primary antibody binding and 2^{nd} antibody binding, including antigen binding in some cases such as Capture ELISA, into to a one step reaction in an immuno-detection. Many washing steps in a regular immunoassays are eliminated or are unnecessary.

In one embodiment, a Non-specific Competitor/Primary antibody Combination comprising a Non-specific Competitor and Primary antibody is used in an immuno-detection which combines blocking and primary antibody binding into a one step reaction.

In another embodiment, a Non-specific Competitor/Specific Indicator Combination comprising a Non-specific Competitor and a Specific Indicator described below is used in an immuno-detection which combines blocking and 2^{nd} antibody binding into a one-step reaction.

In another embodiment, a solution comprising a Non-specific Competitor, a primary antibody and a Specific Indicator described below is used in an immuno-detection which combines blocking, primary antibody binding and a 2^{nd} antibody binding into a one step reaction.

In all the above combinations, the combination can further combine an antigen to capture an antibody in some immuno-detections such as Capture ELISA.

In one embodiment, a Non-specific Competitor/antigen Combination comprising a Non-specific Competitor and an antigen which combines blocking and antigen capturing an antibody into a one-step reaction.

### Specific Indicator and Specific Indicator Solution

The Specific Indicator is a pre-labeled protein (protein-conjugates ) or a prelabeled antibody (antibody-conjugate) which is capable of specifically recognizing a primary antibody or a receptor protein to form a complex without interfering with the primary antibody's antigen-binding or the receptor's ligand-binding capacity.

The label is a protein or a molecule, which directly or indirectly play as a visible or detectable reporter. Examples of the label are colloidal gold, peroxidase, alkaline phosphatase, beta galactosidase, beta-aminase, rhodamine, biotine, avidin, luminase, fluorescent markers and radioisotope

The protein or antibody of the Specific Indicator can be native or recombinant, and the antibody can be either polyclone or monoclone, or single chain generated from any animals or culture cells including hybridoma or bacteria phage display system.

In one embodiment, the protein or antibody of the Specific Indicator is a protein or antibody which specifically recognizes a primary antibody's or a receptor's any portion (native or artificial portion including tag) except its antigen-binding site or ligand-binding site. For example, protein L specifically recognizes the kappa chain of antibody without interfering its antigen-binding capacity.

In another embodiment, the protein or antibody of in the Specific Indicator is a protein or antibody which specifically recognizes an antibody's or a Fc-fusion protein's Fc portion. Examples are Protein A, protein G, Protein A/G, Fc receptor proteins such as CD64, and any antibodies against the Fc portion of any type immunoglobulin, such as, anti-rabbit IgG-Fc antibody, anti-mouse IgG-Fc antibody, anti-human IgE-Fc antibody, and anti-human IgM-Fc antibody.

In another embodiment, in the case of capture ELISA, the protein or antibody of in the Specific Indicator is a protein or antibody which specifically recognizes primary antibody but not capture antibody.

In addition, the Specific Indicator can be a single type of pre-labeled protein (protein-conjugates), e.g., protein-HRP or a pre-labeled antibody (antibody-conjugate) such as anti-rabbit IgG-Fc-HRP, as well as a mixture of different types of pre-labeled proteins and/or pre-labeled antibodies, e.g., a mixture of anti-mouse IgG-Fc-HRP and anti-rabbit IgG-Fc-HRP, or a mixture of anti-mouse IgG-Fc-HRP and Protein A-HRP.

The Specific Indicator Solution is prepared in a solution that optimizes antigen-antibody binding kinetics. An appropriate solution is an aqueous solution or buffer. The solution is preferably provided under conditions that will promote specific binding, minimize non-specific binding, solubilize the protein, stabilize and preserve reagent reactivity, and may contain buffers, detergents, solvents, salts, chelators, proteins, polymers, carbohydrates, sugars, and other substances known to those skilled in the art.

In one embodiment, the Specific Indicator Solution is a solution containing the Specific Indicator and buffer.

The buffer may be selected in accordance with the substance to be analyzed. As such a buffer, one having an appropriate ion concentration and pH which does not inactivate the substance to be analyzed and does not inhibit the antigen-antibody reaction may be used. For example, a phosphate buffer or a Tris buffer may be used. The pH of the buffer is around 4 to 10, more preferably around 6 to 8.

In one embodiment, a salt is added to the Specific Indicator Solution at a high concentration to minimize non-specific binding including non-specific interaction among proteins and antibodies in the immunoassay process. The high concentration of a salt is 300 mM-1M. The preferable concentration is around 400 mM-800 mM. The preferable salt is NaCl or KCl.

The concentration of Specific Indicator in the Specific Indicator Solution is around 0.01-50 ug/ml.

The use of the Specific Indicator has make it possible to combine at least any two of the steps of blocking, primary antibody binding and 2^{nd} antibody binding, including antigen binding in some cases such as Capture ELISA, into to a one step reaction in an immuno-detection, as well as combine all these steps of blocking, a primary antibody binding, a 2^{nd} antibody binding including antigen binding into a one step reaction in an immuno-detection. Many washing steps in a regular immunoassay are eliminated or are unnecessary.

In one example, a testing antigen, an IgG, can be recognized by the Specific Indicator.

In one embodiment, a Specific Indicator/Primary antibody Combination comprising a Specific Indicator and a primary antibody is used in an immuno-detection which combines primary antibody binding and 2^{nd} antibody binding into a one step reaction.

In another embodiment, a Non-specific Competitor/Specific Indicator Combination comprising a Non-specific Competitor and a Specific Indicator is used in an immuno-detection which combines blocking and 2^{nd} antibody binding into a one step reaction.

In another embodiment, a solution comprising a Non-specific Competitor, a primary antibody and a Specific Indicator described below is used in an immuno-detection which combines blocking, primary antibody binding and a 2^{nd} antibody binding into a one step reaction.

In all the above combinations, the combination can further combine an antigen to be captured by an immobilized capture antibody in some immuno-detections such as Capture ELISA, including that a Specific Indicator can combine with a Non-specific Competitor, a primary antibody and an antigen.

In one embodiment, a high concentration of salt is added to the solution of the above combinations. The high concentration of a salt is 300 mM-1M. The preferable concentration is around 400 mM-800 mM.

### Cofactor

The Cofactor is a protein that can catalyze or accelerate or stabilize the interaction among an antigen, a primary antibody and a 2^{nd} antibody, or a receptor and a ligand, such as forming a stable complex of 2^{nd} antibody-conjugate (or Specific Indicator)-primary antibody-detecting antigen (target).

Examples of cofactors are Heat Shock Protein 70 (I1SP 70) and Heat Shock Protein 60 (HSP 60).

The concentration of a Cofactor is around 0.01-3 ug/mL.

### Protease Inhibitor

In case the testing sample (such as cell lysate) containing a protease, a Protease Inhibitor may be added to a One-Step Reaction Solution, or other Combination solutions, to protect antibodies or antibody-complexes or receptors, ligands or receptors-ligands complexes from degradation, especially for the store and reuse of a used One-Step Reaction Solution.

The concentration of a Protease Inhibitor used in this invention is around 1 μM-10 mM.

### One-Step Reaction Solution

The One-step Reaction Solution is a reagent, which functionally be able to combine the 4 steps required in regular immunoassay procedure, blocking, primary antibody binding, washing and 2^{nd} antibody binding into a simple one-step reaction, which procedurally able to combine at least any two of the steps of blocking, primary antibody binding and 2^{nd} antibody binding into to a one step reaction, as well as to combine all the three steps into a one step reaction. In some cases, e.g., Capture ELISA, this invention is to replace blocking, antigen binding to capture antibody (or similarly, receptor binding to ligand), washing, primary antibody binding, washing and 2^{nd} antibody binding with a one-step reaction.

The One-step Reaction Solution comprises 1) a Non-specific Competitor; 2) a Specific Indicator; optional 3) a Cofactor and optional 4) a Protease Inhibitor.

The One-step Reaction Solution is prepared in a solution that optimizes antigen-antibody binding kinetics. An appropriate solution is an aqueous solution or buffer. The solution is preferably provided under conditions that will promote specific binding, minimize non-specific binding, solubilize the protein, stabilize and preserve reagent reactivity, and may contain buffers, detergents, solvents, salts, chelators, proteins, polymers, carbohydrates, sugars, and other substances known to those skilled in the art.

In one embodiment, the One-step Reaction Solution is a buffer solution.

The buffer may be selected in accordance with the substance to be analyzed. As such a buffer, one having an appropriate ion concentration and pH which does not inactivate the substance to be analyzed and does not inhibit the antigen-antibody reaction may be used. For example, a phosphate buffer or a Tris buffer may be used. The pH of the buffer is around 4 to 10, more preferably around 6 to 8.

In one embodiment, a salt is added to the One-step Reaction Solution at a high concentration to minimize non-specific binding including non-specific interaction or among proteins and antibodies in the immunoassay process. The high concentration of the total salt (s) is around 300 mM-2M. The preferable concentration is around 400-800 mM.

The One-step Reaction Solution can be prepared by mixing each of the dry components in one buffer or mixing each of the component solutions or by the combination thereof.

The One-step Reaction Solution is employed in an immuno-detections, whether qualitative or quantitative.

### Primary Antibody

The primary antibody is an antibody which can be specifically recognized by the Antibody Specific Indicator. The primary antibody includes a native or a recombinant antibody, a polyclone or a monoclone or a single chain antibody generated from animal or culture cells including hybridoma or bacteria phage display system.

Examples of primary antibodies are the primary antibodies generated from rabbit and mouse, which specifically against testing antigens.

Affinity purified primary antibodies are preferred.

A primary antibody can be purchased from the market or self-generated by a user.

### Primary Antibody-Supplemented One-Step Reaction Solution

The Primary Antibody-supplemented One-step Reaction Solution comprising 1) a Non-specific Competitor; 2) an Antibody Specific Indicator; 3) a primary antibody; optional 4) a Cofactor; and optional 5) a Protease Inhibitor.

The Primary Antibody-supplemented One-step Reaction Solution is prepared in a solution that optimizes antigen-antibody binding kinetics. An appropriate solution is an aqueous solution or buffer. The solution is preferably provided under conditions that will promote specific binding, minimize non-specific binding, solubilize the protein, stabilize and preserve reagent reactivity, and may contain buffers, detergents, solvents, salts, chelators, proteins, polymers, carbohydrates, sugars, and other substances known to those skilled in the art.

In one embodiment, the Primary Antibody-supplemented One-step Reaction Solution is a buffer solution.

The buffer may be selected in accordance with the substance to be analyzed. As such a buffer, one having an appropriate ion concentration and pH which does not inactivate the substance to be analyzed and does not inhibit the antigen-antibody reaction may be used. For example, a phosphate buffer or a Tris buffer may be used. The pH of the buffer is around 4 to 10, more preferably around 6 to 8.

In one embodiment, a salt is added to the Primary Antibody-supplemented One-step Reaction Solution at a high concentration to minimize non-specific binding including non-specific interaction or among proteins and antibodies in the immunoassay process. The high concentration of the salt is around 300 mM-2M. The preferable concentration is around 500-800 mM.

The Primary Antibody-supplemented One-step Reaction Solution can be prepared by mixing each of the dry components in one buffer or mixing each of the component solutions or the combination thereof.

In one embodiment, the Primary Antibody-supplemented One-step Reaction Solution is prepared in situ wherein a primary antibody is added to a One-step Reaction Solution before the immuno-detection.

In another embodiment, the Primary Antibody-supplemented One-step Reaction Solution is prepared during the preparation of a One-step Reaction Solution to which a primary antibody is added and stored with the One-step Reaction Solution or assembled in the immuno-detection kits.

In one embodiment, the Primary Antibody-supplemented One-step Reaction Solution is prepared by adding a primary antibody to a Non-specific Competitor/Specific Indicator Combination.

In another embodiment, the Primary Antibody-supplemented One-step Reaction Solution is prepared by adding a Non-specific Competitor to a Specific Indicator/Primary antibody Combination.

In another embodiment, the Primary Antibody-supplemented One-step Reaction Solution is prepared by adding a Specific Indicator to the Non-specific Competitor/Primary antibody Combination.

In another embodiment, the Primary Antibody-supplemented One-step Reaction Solution further combines an antigen in some immuno-detections, e.g. Capture ELISA.

The Primary Antibody-supplemented One-step Reaction Solution is employed an immuno-detection, whether qualitative or quantitative.

The Primary Antibody-supplemented One-step Reaction Solution is recoverable from an immuno-detection and is reusable.

### Rapid Wash Solution

The Rapid Wash Solution is a buffer solution containing a high concentration of salt. The high concentration of the salt is around 300-1M, preferable 0.4-800 mM. The preferred salt is NaCl or KCl.

The preferred buffer is any buffer with pH 4-10 and the preferred buffer is Phosphate buffer or Tris buffer with pH is 6-8.

The Rapid Wash Solution can efficiently remove antibody's non-specific binding from the immuno-detection system to reduce wash times and washing time.

### Universal Rapid Immuno-Detection

The Universal Rapid Immuno-detection method is a method suitable for various immuno-detections, including conventional immunoassays based on antibody-antigen binding mechanism, e.g., Immuno-blot (Western Blot, Dot blot), ELISA, IHC and ICC, as well as other assays based on the mechanisms similar to the antibody-antigen binding mechanism, e.g., a receptor-ligand assay.

The Universal Rapid Immuno-detection method comprising the steps of: 1) one-step reaction; 2) washing; and 3) developing.

The one-step reaction is a combination of blocking, primary antibody binding to antigen (or receptor binding to ligand), specific indicator binding to primary antibody (receptor) in a same time period. In some cases, e.g., Capture ELISA, the one-step reaction is a combination of blocking, antigen binding to capture antibody (or similarly, receptor binding to ligand), primary antibody binding, and 2^{nd} antibody binding into a one-step reaction where washings between each step are eliminated or are unnecessary.

The One-step Reaction Solution is reacted for a sufficient amount of time to allow the antibody to react and bind to the protein to form an antibody-antigen complex. The shortest amount of reaction time that results in binding is desired to minimize the time required to complete the assay. An appropriate reaction time period for most immuno-detection is around 30 minutes to 1 hour. In some cases, only 1 minute is needed for the reaction.

The One-step Rapid Reaction is performed at any temperature at which the reagents do not degrade or become inactivated. A temperature between approximately 15° C. and 40° C. is preferred, and most preferred reaction temperature is ambient or room temperature.

In one embodiment, the one-step reaction or binding step in the Universal Rapid Immuno-detection method is accomplished by incubation of Primary Antibody-supplemented One-step Reaction with testing sample immobilized on a solid phase. In one embodiment of the invention, the Universal Rapid Immuno-detection of the protein comprises the steps of:
1) incubating the testing sample on a solid phase with the Primary Antibody-supplemented One-step Rapid Reaction Solution;
2) washing the solid phase;
3) developing color.

The substance to be analyzed in the analyzing method of the present invention is not particularly limited, so long as it is a substance (particularly a physiologically active substance) which may be generally analyzed by the use of the antigen-antibody reaction or similar mechanism reaction. Examples are antigens, antibodies, receptors, ligands.

The Universal Rapid Immuno-detection is employed in an immuno-detection, whether qualitative or quantitative.

The concentration of the protein in the sample is determined by conventional detection methods used in immuno-detection, such as by comparing the intensity of the color produced by the sample to a color card or by using a reflectometer or using microplate reader.

In other embodiments, the test sample is immobilized on the solid phase such as NC membrane, surface of microplate or glass slid.

### Immunoassay Kit

The Non-specific Competitor or solution, the Specific Indicator or solution, the Antibody-supplemented One-step Reaction Solution, the Non-specific Competitor/Specific Indicator Combination or Solution, the Non-specific Competitor/Primary antibody Combination or Solution, the Specific Indicator/Primary antibody Combination or Solution, or any Combinations according to this invention, may be assembled in a kit with conventional immuno-detection reagents for detection of the protein. The kit may contain a standard for quantification reference or controlling assay self's performance. The kit containing these reagents provides a simple and rapid on site detection of the protein.

The One-step Reaction Solution described above is used as the basic reagents of a number of different immunoassays to identify or quantify a specific protein in a sample.

In one embodiment, the invention provides a kit for the identification and quantification by the immunoassay method comprising:
1) a means of extracting the protein from a sample;
2) a solid support to immobilize the testing sample;
3) a One-step Reaction Solution.

In one embodiment, the reagents, including the antibody are dry.

In another embodiment, the kit further comprises a substrate for the developing.

The kit may additionally contain an equipment for obtaining the sample, a vessel for containing the reagents, a timing means, a buffer for diluting the sample, and a colorimeter, reflectometer, or standard against which a color change may be measured.

The reagents, immunoassay methods, kits and uses of the reagents described above will be further understood with reference to the following non-limiting examples. The examples below show typical experimental protocols and reagents that can be used in the detection of specific protein. Such examples are provided by way of illustration and not by way of limitation.

Numerous references cited above are all incorporated herein in their entireties.

### EXAMPLES

### Materials

### 1. Non-specific Competitor Solution

Dissolve 10 to 100 g of the non-specific protein to I liter buffered solution containing: 5-50 mM Sodium EDTA, 0.3-0.5M NaCl, 1-5 mg/ml MgCl2, 0.05% Sodium Azide, 10-100 mM Tris-HCl buffer (or phosphorate buffer or the PBS), pH7-8.

Store the Non-specific Competitor Solution at 2˜4 ° C. before use.

### 2. Specific Indicator Solution

Make 1 liter of the following solution: 0.1% BSA, 0.1% potassium sorbate, 5 mM EDTA, 5 uM protease inhibitor, 0.5 M NaCl in PBS buffer, then taking 10ml of this solution and gentle mixing it with 5˜50 ug (or 30˜300 ug for IHC or ICC ) goat anti-rabbit IgG-Fc antibody and 5˜50 ug (or 30˜300 ug for IHC or ICC) goat anti-mouse IgG-Fc antibodies. (10 ml is sufficient for detection of one 96-well ELISA plate or a piece of 60˜100 square centimeter Western the Blot membrane or 50 IHC/ICC slides). Note: Selected Specific Indicator must be able to recognize primary antibody that will be used in the detection system. Addition of other reagents such as a Protease inhibitor or a Cofactor described in the invention is optional.

Store the Specific Indicator Solution at 2˜4° C. before use.

### 3. One-Step Rapid Reaction Solution

Take 10 ml of the above Non-specific Competitor Solution and gently mix it with 50 nmol protease inhibitor, 1-50 ug Specific Indicator (or 30˜300 ug for IHC or ICC ) (e.g., 1˜50 ug goat anti-rabbit IgG-Fc antibody and 1˜50 ug goat anti-mouse IgG-Fc antibodies).

10 ml is sufficient for detection of one 96-well ELISA plate or a piece of 60˜100 square centimeter Western the Blot membrane or 50 IHC slides. Note: Selected Specific Indicator must be able to recognize primary antibody that will be used in the detection system. Addition of other reagents such as a Protease Inhibitor or a Cofactor described in the invention is optional.

Store the One-step Reaction Solution at 2˜4° C. in refrigerator before use.

### 4. Rapid Wash Solution

Dissolve 0.3-0.5 mol NaCl into 1 liter of 10˜100 mM with pH7˜8 phosphorate buffer (or the Tris- hydrochloric acid buffer, or PBS), add Tween-20 to 0.01-0.03%.

Store the Wash Solution at the room temperature.

### 5. Substrate

1) For Western blot or Dot blot or IHC/ICC: if the antibody-specific indicator is labeled with peroxidase (HRP), use Chemiluminescent (e.g., Amersham Biosciences's ECL Detection kit) or 3,3′-Diaminobenzidine (DAB) (e.g. example, SIGMA's product, D4418); if the antibody-specific indicator is labeled with alkalinity phosphatase (AP) use BCIP/NBT (e.g., SIGMA's product, B5655).
2) For ELISA: if the antibody-specific indicator is labeled with HRP, use 3,3′, 5,5′ the—TMB liquid as substrate (e.g., SIGMA T8665); if the antibody-specific indicator is labeled with alkalinity phosphatase (AP), use pNPP (e.g., SIGMA N1891).

### 10. Stop Solution

### ELISA: 0.5M H2SO4 or 1N HCL.

### Methods

### 1. Rapid Western Blot or Dot Blot Detection:

1) Take 5 to 20 ug of a primary antibodies specifically against the testing antigen and mix it with 10 ml of the invention described “One-step Reaction Solution”; directly overlay the pre-prepared sample containing membrane (appropriate 8×10 cm²) (described in note 1 below) with the primary antibody-supplemented One-step Reaction Solution, and incubate with gently shaking at room temperature for 10-30 minute.
Note 1: The sample preparation is the same comparing to the regular method. In general, testing protein (antigen) is dot blotted or Western-transferred onto nitrate-cellulose (NC) membrane or onto PVDF membrane after SDS-PAGE separation.
Note 2: The solution must cover the membrane.
2) (Option) Recover the primary antibody-supplemented One-step Reaction Solution for reuse, but only good for testing same antigen, (generally can be re-used for 5 times).
3) Wash the membrane one time for one minute with 50 ml of the invention described “Rapid Wash Solution” and rinse it two times (10 seconds each time) with 100 ml of 10× diluted Rapid Wash Solution or distilled water.
Develop color (this step is the same comparing to the regular method) on the membrane by incubating with a suitable substrate described in the “Materials” until desired color appears.
Note 3: If not using Chemiluminescent as a substrate, once a desired color appears, wash the membrane with sufficient distilled water to prevent from over-development.

### 2. Rapid Indirect ELISA:

1) Mix 5 to 20 ug of primary antibodies specifically against the testing antigen with 10 ml of the invention described “One-step Reaction Solution”, then add 100 ul of the primary antibody-supplemented one-step rapid reaction solution to each antigen-coated well or blank well of the testing plate (described in note 1 below) and incubate with or without gently shaking at room temperature for 10-30 minute.
Note 1: The sample preparation is the same comparing to the regular ELISA method. In general, the testing protein (antigen) is coated onto the well of a 96-well ELISA plate with amount of 100 ul/well. Each plate must remain a certain wells coated with buffer alone as a negative control.
2) (Option) Recover the primary antibody-supplemented One-step Reaction Solution for reuse, but only good for testing same antigen, (generally can be re-used for 5 times).
3) Wash wells three times with 200 ul/well of the invention described “Rapid Wash Solution”.
4) Develop color (this step is the same comparing to the regular method) on the wells by adding 100 ul/well of a suitable substrate (TMB or pNPP) as described in the “Materials”. After desired colors appear add 100 ul/well stop solution (0.5M H2SO4), and then read the OD with a microplate reader at 450 nm (for TMB substrate) or at 405 nm (for pNPP substrate).

### 3. Rapid Immunohistochemistry (IHC) or Immunocytochemistry (ICC)

1) Mix 4 to 30 ug of a primary antibody specifically against the testing antigen with 2 ml (sufficient for 10 slides) of the invention described “One-step Reaction Solution”; directly overlay the sample containing slides (described in notel below) with 200 ul/slide of the primary antibody-supplemented One-step Reaction Solution, and incubate in a humility incubator at 37° C. or room temperature for 10-30 minutes.
Note 1: The sample preparation is the same comparing to the regular IHC or ICC method. In general, samples (tissue or cells) on slides must be fixed and pre-treated (in necessary) to inactivate the endogenous peroxidase.
Note 2: The solution must cover the membrane.
2) (Option) Recover the primary antibody-supplemented One-step Reaction Solution for reuse, but only good for testing same antigen, (generally can be re-uses for up to 5 times).
3) Rinse slides 3 times with 2 ml of the invention described “Rapid Wash Solution” and one time with 3 ml of 10× diluted Rapid Wash Solution or distilled water.
4) Develop color (this step is the same comparing to the regular method) on the section with a suitable substrate described in the “Materials” for 1-10 min until a desired color appears and then stop the development by rinse slides with distilled water.
5) Counterstaining (this step same to the regular method): incubate the sides in a solution of Mayer's hermatoxyin for 0.5-5 min at room temperature, then gently wash the slides with distilled water before taking photo or mounting the sections with glycerol gelatin and sealing coverslips with clear nail polish.

### EXAMPLES

### Example 1

### Examination of the Blocking Capacity of Non-specific Competitors in Rapid Dot Blot Detection

### Material:

Binding buffer: 30 mM Sodium EDTA, 0.5M NaCl, 5 mg/ml MgCl2, 0.05% Sodium Azide, 50 mM Tris-HCl buffer, pH8,
Wash buffer: 50 mM Tris-HCl buffer, pH8, 0.5M NaCl, 0.3% Tween-20.
Non-special competitor solution: prepare following non-specific competitor solution with above binding buffer, 5 ml each: A: binding buffer alone (without non-specific competitor), B: 3% BSA, C: 10 mg/ml BSA, D: 10 mg/ml Non-fat milk powder, E: 10% Egg white, F: 10 mg/ml Goat IgG, G: 10% Goat serum, and H: 10 mg/ml pork skin gelatin.
Antigen: purified normal rabbit IgG, 1 ug/ul.
Antibody: goat anti-rabbit IgG-HRP conjugate.
Substrate: Diaminobenzidine (DAB) (SIGMA's product, D4418).
Nitrate-cellulose (NC) membrane.
   Sample treatment:
   Dot blotted 1 ul of the rabbit IgG (1 ug/ul, as antigen here) onto nitrate-cellulose (NC) membrane. Make 8 pieces of the membrane with two dots per piece, and label them with letters, A to H.

### Immuno-Detection:

1. For comparison, the dot-blotted NC membrane Piece B was incubated with 3% BSA non-specific solution for 1 hour and then washed three time with wash buffer, following incubation with detect antibody, lug/ml of goat anti-rabbit IgG-HRP conjugate, in binding buffer for 30 min at room temperature.
2. To each 5 ml of non-specific competitor solutions (A, and C to H), added 5 ug of goat anti-rabbit IgG-HRP conjugate (final concentration, 1 ug/ml) and then directly incubated them with the corresponding dot-blotted NC membranes (pieces A, and C to H), respectively, for 30 min with gently shaking at room temperature.
3. All membranes (pieces A to H, from step 1 and step 2) were washed with 5 ml of rapid wash solution for one minute, and then rinse them twice (10 seconds each time) with 100 ml distilled water.
4. After washes, membranes were incubated with DAB substrate solution for color development till the desired color appears.

### Result:

As showed in the FIG. 1, in this experiment, dot blots were detected using two different methods, with a separated blocking step (regular method) and with a combination of blocking and antibody binding (rapid method). The results indicated that without using a Non-specific Competitor (a negative control, A), the blot is very dark and not detectable due to a high non-specific binding; in contrast, by using Non-specific Competitors (C-J), the non-specific background binding was dramatically reduced to a similar level as pre-blocked blot (B.). This result demonstrated that the combination of blocking and antibody binding into one-step for an immuno-detection using Non-specific Competitor solutions achieved the same result comparing to the regular procedure.

### Example 2

### Examination of the Combination of Primary Antibody Binding and 2^{nd} Antibody Binding into a One-Step Reaction

### Material:

Binding buffer: 30 mM Sodium EDTA, 0.5M NaCl, 5 mg/ml MgCl2, 0.05% Sodium Azide, 50 mM Tris-HCl buffer, pH8,
Wash buffer: 50 mM Tris-HCl buffer, pH8, 0.5M NaCl, 0.3% Tween-20.
Blocking solution: 3% BSA in binding buffer.
Antigen: beta-galactosidase.
Primary antibody: Mouse IgG against beta-galactosidase.
2^{nd} Antibody: Goat anti-mouse IgG (H+L)-HRP conjugate.
Specific indicator: Goat anti-mouse IgG Fc-HRP conjugate.
Substrate: Diaminobenzidine (DAB) (SIGMA's product, D4418).
Nitrate-cellulose (NC) membrane.

### Sample Treatment:

Testing antigen beta-galactosidase at three different concentrations: 0.3, 0.1 and 0.03 ug/ul. were dot-blotted (2 ul/dot) onto two pieces of nitrate-cellulose (NC) membrane.

### Immuno-Detection:

1. The dot-blotted NC membranes were blocked by incubation with blocking solution for one hour at room temperature, and then washed three times with wash buffer.

2. Antibody bindings were carried out by following two different methods: 1), Combination of 1^{st} antibody binding and 2^{nd} antibody binding: To 10 ml of the binding buffer added and mixed with both 10 ug of primary antibody and 10 ug of Specific indicator, and then incubation this solution with the pre-blocked NC membrane (membrane 1) for 30 min with gently shaking at room temperature. 2), Separated 1^{st} antibody binding and 2^{nd} antibody binding (regular method): To two 10-ml binding buffer added 10 ug of primary antibody and 10 ug of 2^{nd} antibody, respectively; Incubated the pre-blocked NC membrane (membrane 2) with the primary antibody-supplemented binding solution for 60 min at room temperature; Wash the membrane three times with wash solution, and then incubated it with the 2^{nd} antibody-supplemented binding solution for another 60 min at room temperature.

3. Both membranes (membrane 1 and 2) were washed with 5 ml of wash solution for one minute, and then rinse them twice (10 seconds each time) with 100 ml distilled water.
4. After washes, membranes were incubated with DAB substrate solution till desired color developed.

### Result:

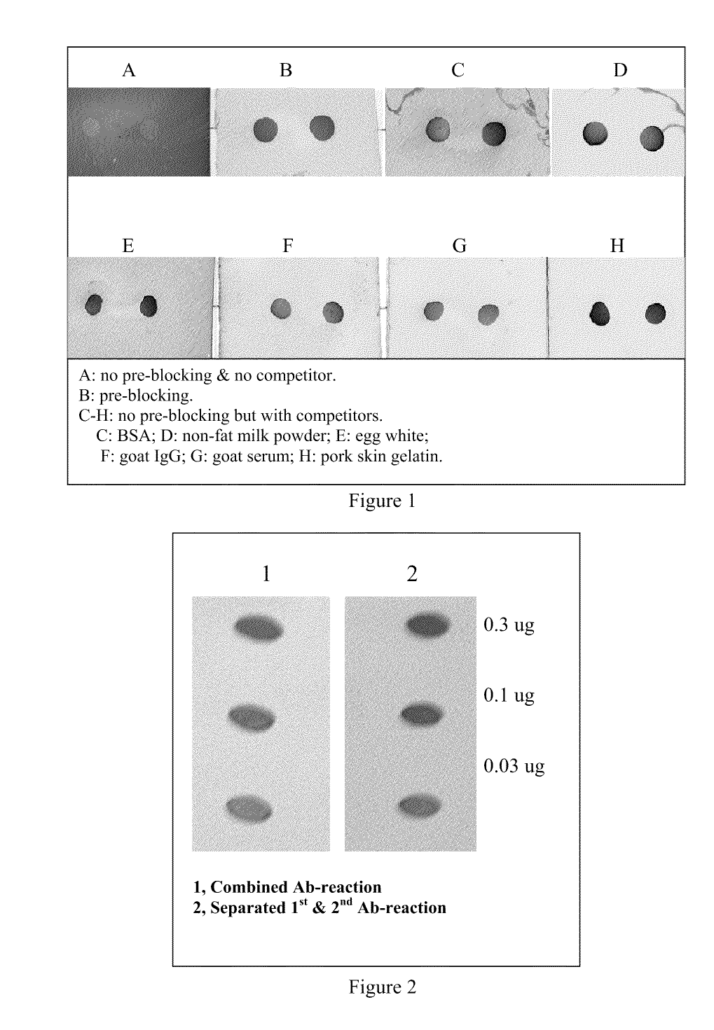

As showed in FIG. 2, with using Fc-specific 2^{nd} antibody the one-step antibody reaction by combining 1^{st} antibody binding with 2^{nd} antibody binding worked well as separated two steps antibody reactions (1^{st} antibody binding following 2^{nd} antibody binding). This result demonstrated that combination of primary antibody binding and 2^{nd} antibody binding into one-step for an immuno-detection using Specific indicator, Fc-specific 2^{nd} antibody, achieved the same result comparing to the regular procedure.

### Example 3

### Rapid ELISA Using Different Types of 2^{nd} Antibodies

### Material:

Coating buffer: 50 mM carbonate-bicarbonate buffer pH9.0.
One-step Rapid Reaction Solution: 50 mM Tris-HCl buffer, pH8.0, 30 mM sodium-EDTA, 0.5M NaCl, 0.05% Sodium Azide, 50 ug/ml Ampicillin, 5 mg/ml MgCl, 5% fish gelatin, 0.1 ug/ml Heat shock protein-70, 0.5 ug/ml Goat anti-rabbit IgG Fc-HRP conjugate and 0.5 ug/ml goat anti-mouse IgG Fc-HRP conjugate.
Non-specific Competitor Solution: 30 mM Sodium EDTA, 0.5M NaCl, 5 mg/ml MgCl2, 0.05% Sodium Azide, and 10 mg/ml BSA in 50 mM Tris-HCl buffer, pH8,
Rapid Wash Solution: 50 mM Tris-HCl buffer, pH8, 0.5M NaCl, 0.2% Tween-20.
Antigen: beta-galactosidase.
Primary antibody: Mouse IgG against beta-galactosidase.
2^{nd} Antibody: Goat anti-mouse IgG (H+L)-HRP conjugate.
Substrate: 3,3′, 5,5′ Tetramethylbenzine (TMB) liquid substrate system (SIGMA T8665).
96-well Microtiter ELISA plate.

### Sample Treatment:

Coating: 100 ul of antigen, beta-galactosidase, which was dissolved in coating buffer at a series of eight different concentrations made by 3× dilution from 500 ng/mi to 0.7 and 0.00 ng/ml, was added to each well of a 96-well ELISA plate and incubated for two hours, and then removed coating solution, wash each well with 150 ul of wash buffer for 3 times.
Antibody reaction solution: three groups of antibody reaction solutions were prepared as following: A), 5 ml of One-step rapid reaction solution plus 5 ug of primary antibody (mouse antibody against beta-galactosidase); B), 5 ml of One-step rapid reaction solution alone without primary antibody; and C), 5 ug of primary antibody (mouse antibody against beta-galactosidase) and 5 ug of 2^{nd} antibody (goat anti-mouse whole IgG-HRP) dissolved in 5 ml of non-specific competitor solution.

### Immuno-Detection (Rapid Indirect ELISA):

1. Added 100 ul/well of each prepared antibody reaction solutions (A, B and C) to every series of the antigen-coated wells without pre-blocking, and incubation for 30 min at room temperature
2. Removed the antibody reaction solutions and washed wells three times with 200 ml/well of wash buffer.
3. Added 100 ul/well of the substrate (TMB) and incubated for 1˜5 min till desired color developed, added 100 ul/well stop solution (0.5M H2SO4) to terminate color development and then read the OD with a microtiter plate reader at 450 nm.

### Results:

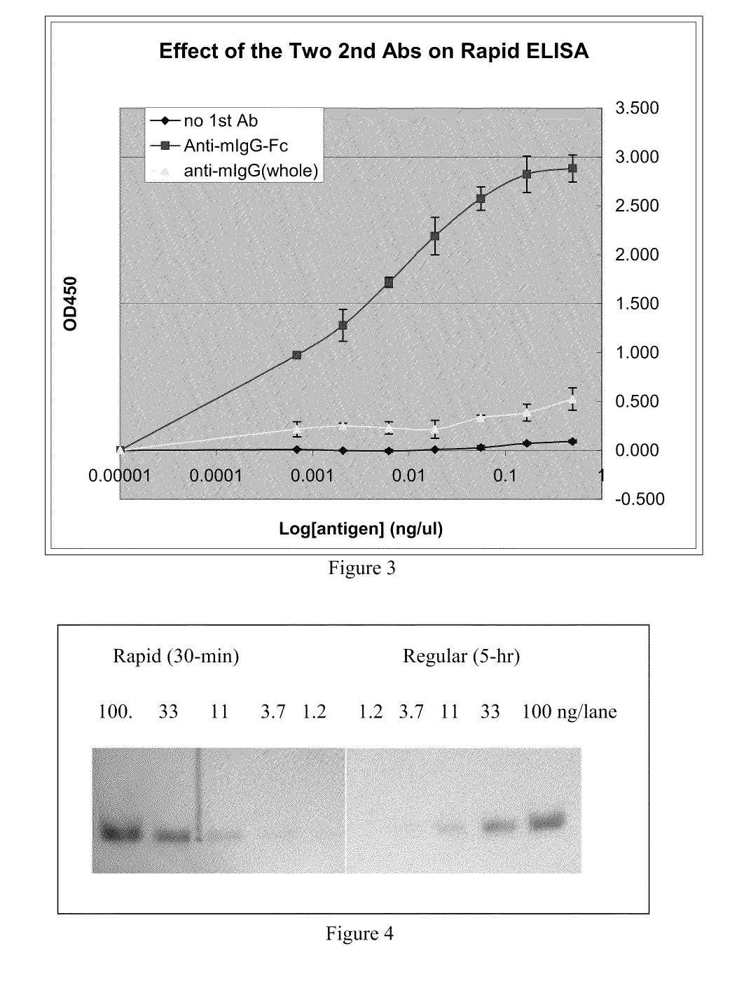

As showed in the FIG. 3, the Fc-specific 2^{nd} antibody worked well in the rapid indirect ELISA while the normal 2^{nd} antibody showed a little signal, and without primary antibody, the Fc-specific 2^{nd} antibody alone did not make any signal. This suggested that: 1) the normal 2^{nd} antibody interfere primary antibody binding capacity, therefore it is not good for Rapid immunoassays, even though it is good for a regular ELISA, and 2) the Fc-specific 2^{nd} antibody is a primary antibody-dependent indicator, not non-specifically bind to antigen or other proteins, therefore it is a good antibody-specific indicator for Rapid immuno-detection.

### Example 4

### Comparison of Rapid Immuno-Detection with Regular Immuno-Detection for Western-blot

### Material:

One-step Rapid Reaction Solution: 50 mM Tris-HCl buffer, pH8.0, 30 mM sodium EDTA, 0.5M NaCl, 0.05% Sodium Azide, 50 ug/ml Ampicillin, 5 mg/ml MgCl, 5% fish gelatin, 0.1 ug/ml Heat shock protein-70, 10 uM E64, 5% Glycerol, 1 ug/ml Protein A-HRP conjugate and 0.5 ug/ml goat anti-mouse IgG Fc-HRP conjugate.
Rapid Wash Solution: 50 mM Tris-HCl buffer, pH8, 0.5M NaCl, 0.3% Tween-20.
Blocking buffer (for a regular immuno-detection): PBS with 3%-nonfat milk powder.
Buffer for regular immuno-detection: PBS with 0.05% Tween20
Antigen: GST protein.
Primary antibody: Rabbit IgG against GST.
2^{nd} Antibody: Goat anti-rabbit whole IgG-HRP conjugate.
Substrate: Chemiluminescent (Amersham Biosciences's ECL Detection kit).
Nitrate-cellulose (NC) membrane (8×10 cm2).

### Sample Treatment:

Serially diluted GST proteins were loaded into a 10% mini SDS-gel at indicated amount (1.2˜100 ng per well) and Western-blotted onto a nitrocellulose membrane after SDS-PAGE. The membrane was then cut into two pieces, one for rapid immuno-detection and the other one for regular immuno-detection.

### Rapid Immuno-Detection:

1. Added 10 ug of the primary antibodies (Rabbit IgG anti-GST) to 10 ml of the one-step rapid reaction solution; directly overlay the blotted NC membrane (without pre-blocking) with the primary antibody-supplemented one-step rapid reaction solution, and incubate with gently shaking at room temperature for 30 minute. Note: the solution must cover the membrane.
2. (Option) Recover the primary antibody-supplemented one-step rapid reaction solution for reuse in testing same antigen.
3. Washed the membrane one time for one minute with 50 ml of the “rapid wash solution” and rinsed it twice (10 seconds each time) with 100 ml distilled water.
4. Blotted on the membrane was developed for approximately 30 seconds with Chemiluminescent (as substrate) using Amersham Biosciences's ECL Detection kit and according to the manufacture's instruction.

### Regular Immuno-Detection:

1. Incubated the blotted membrane with blocking buffer for 2 hr,
2. Washed the membrane three times with PBS/0.05% Tween20.
3. Incubated the membrane with primary antibody solution (1/1000-diluted rabbit anti-GST antibodies in PBS/0.05% Tween20) for 2 hours at room temperature.
4. Washed the membrane three times with PBS/0.05% Tween20.
5. Incubated the membrane with 2nd antibody solution (1/1000 diluted goat anti-rabbit whole IgG-Peroxidase conjugate in PBS/0.05%Tween20) for 1 hr at room temperature.
6. Washed the membrane three times with PBS/0.05% Tween20.
7. Blotted on the membrane was developed for approximately 30 seconds with Chemiluminescent (as substrate) using Amersham Biosciences's ECL Detection kit and according to the manufacture's instruction.

### Result:

As showed in the FIG. 4, the results by both methods are very similar but the testing time and procedure are great different: 30 minutes and 3 steps (by a rapid method) vs. 5 hours and 7 steps (by a regular method).

### Example 5

### Comparison of Rapid Immuno-Detection with Regular Immuno-Detection for Indirect ELISA

### Material:

One-step Rapid Reaction Solution: 50 mM Tris-HCl buffer, pH8.0, 30 mM sodium-EDTA, 0.5M NaCl, 0.05% Sodium Azide, 50 ug/ml Ampicillin, 5 mg/ml MgCl, 5% fish gelatin, 0.1 ug/ml Heat shock protein-70, 0.5 ug/ml Goat anti-rabbit IgG Fc-HRP conjugate and 0.5 ug/ml goat anti-mouse IgG Fc-HRP conjugate.
Rapid Wash Solution: 50 mM Tris-HCl buffer, pH8, 0.5M NaCl, 0.2% Tween-20.
Blocking buffer (for regular immuno-detection): PBS with 3%-nonfat milk powder.
Buffer for regular immuno-detection: PBS with 0.05% Tween20
Antigen: GST protein.
Primary antibody: Rabbit IgG against GST.
2^{nd} Antibody: Goat anti-rabbit whole IgG-HRP conjugate.
Substrate: 3,3′, 5,5′ Tetramethylbenzine (TMB) liquid substrate system. (SIGMA T8665).
Stop solution: 1N HCl.
96-well microtiter ELISA plate.

### Sample Treatment:

Coating: 100 ul of antigen protein GST, dissolved in PBS buffer at a series of eight different concentrations made by 3× dilution from 30 ng/ul to 0.04 and 0.00 ng/ul, was added to each well of a 96-well ELISA plate and incubated for two hours at room temperature, and then removed the coating solution, wash wells with 200 ul/well of wash buffer for 3 times.

### Rapid Immuno-Detection:

1. Mixed 10 ug of the primary antibodies (Rabbit IgG anti-GST) with 10 ml of the one-step rapid reaction solution and then directly add 100 ul/well to the antigen coated ELISA plate, and then incubated for 30 minute at room temperature.
2. (Option) Recover the primary antibody-supplemented one-step rapid reaction solution for reuse in testing same antigen.
3. Washed the wells 3 time with 200 ml of the rapid wash solution.
4. Developed color in all wells with 100 ul/well TMB solution and waiting till desired color appeared; stopped the color development by adding 100 ul/well 1N HCl, and then read the absorbance at 450 nm in a microplate reader.

### Regular Immuno-Detection:

1. Blocked the pre-coated wells with 200 ul/well blocking buffer for 2 hr.
2. Washed the wells three times with PBS/0.05% Tween20.
3. Incubated the wells with primary antibody solution (1/1000-diluted rabbit anti-GST antibodies in PBS/0.05% Tween20) for 2 hours at room temperature.
4. Washed the wells three times with PBS/0.05% Tween20.
5. Incubated wells with 2nd antibody solution (1/1000 diluted goat anti-rabbit whole IgG-Peroxidase conjugate in PBS/0.05% Tween20) for 1 hr at room temperature.
6. Washed the wells three times with PBS/0.05% Tween20.
7. Developed color in all wells with 100 ul/well TMB solution and waiting till desired color appeared; stopped the color development by adding 100 ul/well 1N HCl, and then read the absorbance at 450 nm in a microplate reader.

### Results:

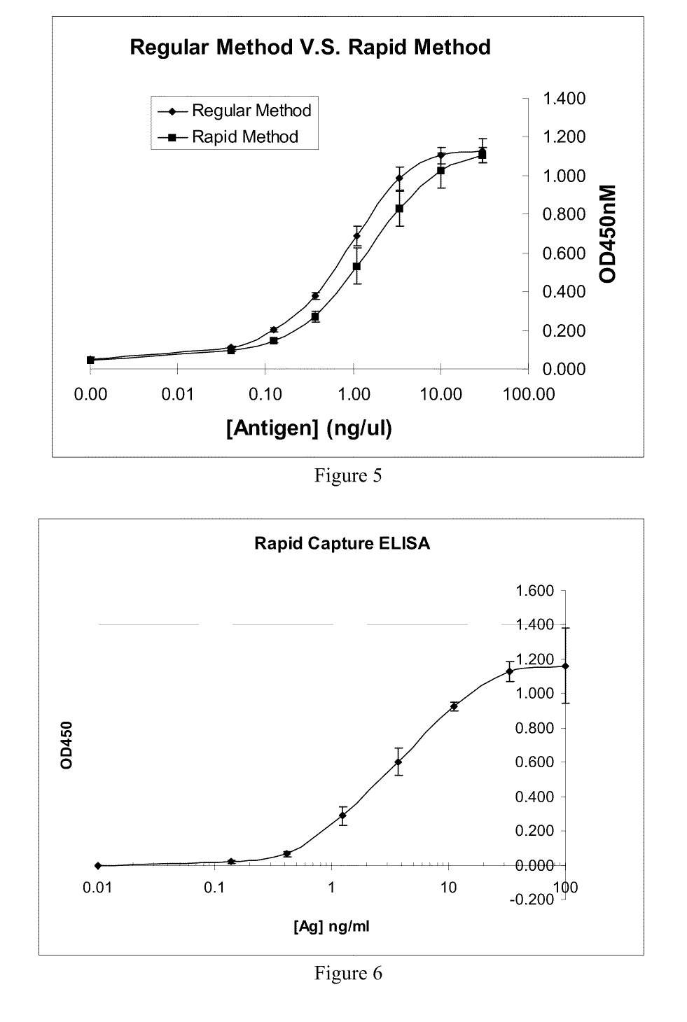

As showed in the FIG. 5, the results by both methods showed comparative sensibility for the rapid method and the regular method. However, by using the rapid method the testing time is much shorter (30 minutes vs. 5 hours) and the assay procedure is much simpler (3 steps vs. 7 steps).

### Example 6

### Universal Rapid Immuno-Detection for Capture ELISA

### Material:

One-step Rapid Reaction Solution: 50 mM Tris-HCl buffer, pH8.0, 30 mM sodium-EDTA, 0.5M NaCl, 0.05% Sodium Azide, 50 ug/ml Ampicillin, 5 mg/ml MgCl, 10 mg/ml BSA, 0.1 ug/ml Heat shock protein-70 and 0.5 ug/ml Rabbit anti-mouse IgG Fc-HRP conjugate.
Rapid Wash Solution: 50 mM Tris-HCl buffer, pH8, 0.5M NaCl, 0.2% Tween-20.
Antigen: human TNF-alpha.
Capture antibody: Rabbit IgG against human TNF-alpha.
Primary antibody: Mouse IgG against human TNF-alpha.
Substrate: 3,3′, 5,5′ Tetramethylbenzine (TMB) (SIGMA T8665).
Stop solution: 1N HCl.
96-well microtiter ELISA plate.

### Sample Treatment:

Coating: 100 ul of 10 ng/ul capture antibody dissolved in 50 mM carbonate-bicarbonate buffer pH9.6, was added to each well of a 96-well ELISA plate and incubated for two hours at room temperature, and then removed the coating solution, washed wells with 200 ul/well of wash buffer for 3 times.

### Rapid Immuno-Detection:

1. a) Mixed 5 ug of the primary antibodies with 10 ml of the one-step rapid reaction solution; b) dissolved human TNF-alpha (testing antigen) with the primary antibody-supplemented one-step rapid reaction solution (made in above step 1-a)) and made 200 ul each of following final TNF-alpha's concentrations: 100 ng/ml, 33 ng/ml, 11 ng/ml, 3.6 ng/ml, 1.2 ng/ml, 0.4 ng/ml, 0.13 ng/ml and 0.00 ng/ml. c) added directly 100 ul/well of the above mixture to the capture antibody-coated wells of ELISA plate, and then incubated for 60 minute at room temperature.
2. Washed the wells 3 time with 200 ml of the rapid wash solution.
3. Developed color in all wells with 100 ul/well TMB solution and waiting till desired color appeared; stopped the color development by adding 100 ul/well 1N HCl, and then read ODs at 450 nm in a microplate reader.

### Results:

As showed in FIG. 6, the experiment demonstrated that the Universal Rapid Immuno-detection method is also suitable for complicate Capture ELISA as well as for indirect ELISA (example 5). In the Capture ELISA, the rapid method combined blocking, antigen binding, primary antibody binding, 2^{nd} antibody binding into a one-step rapid reaction without using a pre-labeled primary antibody (most regular methods for Capture ELISA use a pre-labeled primary antibody).

### Example 7

### Detection of the Binding Capacity of TNF-alpha Receptor Using Universal Rapid Immuno-Detection Method

### Material:

One-step Rapid Reaction Solution: 50 mM Tris-HCl buffer, pH8.0, 30 mM sodium-EDTA, 0.5M NaCl, 0.05% Sodium Azide, 50 ug/ml Ampicillin, 5 mg/ml MgCl, 10 mg/ml BSA, 0.1 ug/ml Heat shock protein-70 and 0.5 ug/ml goat anti-human IgG Fc-HRP conjugate.
Rapid Wash Solution: 50 mM Tris-HCl buffer, pH8, 0.5M NaCl, 0.2% Tween-20.
Capture protein: human TNF-alpha.
Testing protein: human TNF-alpha receptor Fc-fusion: TNFRII-Fc.
Substrate: 3,3′, 5,5′ Tetramethylbenzine (TMB) liquid substrate system.(SIGMA T8665).
Stop solution: 1N HCl.
96-well microtiter ELISA plate.

### Sample Treatment:

Coating: 100 ul/well of 10 ng/ul capture protein (human TNF-alpha) dissolved in 50 mM carbonate-bicarbonate buffer pH9.6, was added to wells of a 96-well ELISA plate and incubated for two hours at room temperature, and then removed the coating solution, washed wells with 200 ul/well of wash buffer for 3 times.

### Rapid Immuno-Detection:

1. Mixed human TNFRII-Fc (TNF-alpha receptor) with one-step rapid reaction solution and made 200 ul each of following final TNFRII-Fc concentrations: 1000 ng/ml, 333 ng/ml, II ng/ml, 37 ng/ml, 12 ng/ml, 4 ng/ml, 1 ng/ml and 0.00 ng/ml; added 100 ul each of the mixture (two assays for each concentration) to the TNF-alpha-coated well, and then incubated for 30 minutes at room temperature.
2. Washed the wells 3 times with 200 ml of the rapid wash solution.
3. Developed color in all wells with 100 ul/well TMB solution and waiting till desired color appeared; stopped the color development by adding 100 ul/well IN HCl, and then read ODs at 450 nm in a microplate reader.

### Results:

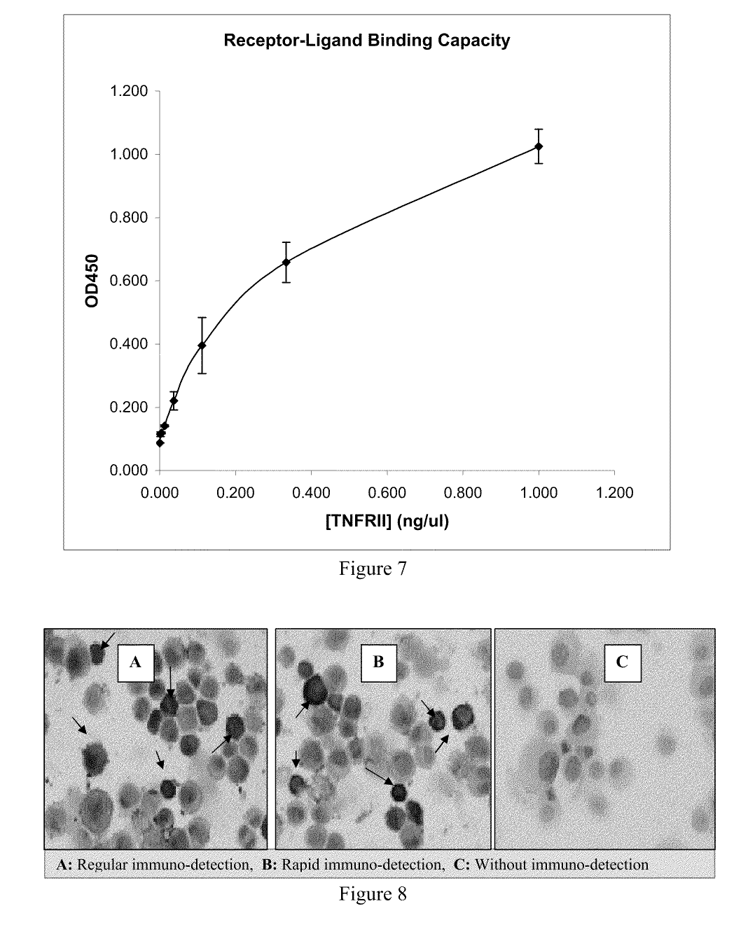

The experiment examined TNF-alpha receptor's ligand-binding capacity at a series concentration using the Universal Rapid Immuno-detection method. The results demonstrated that the Universal Rapid Immuno-detection method is suitable for detecting receptor's ligand-binding capacity. The rapid method successfully combined blocking, receptor-ligand binding, detector antibody binding into a one-step reaction. This experiment is also an example of using a protein with AN artificial portion (in this case the fusion Fc is an artificial portion) which can be recognized by specific indicator in the one-step rapid reaction solution. As shown in FIG. 7, each indicated point in the curves represents an average OD450 of two individual assays with standard deviation showed.

### Example 8

### Comparison of Universal Rapid Immuno-Detection with Regular Immuno-Detection in Immunocytochemistry

### Material:

One-step Rapid Reaction Solution: 50 mM Tris-HCl buffer, pH8.0, 30 mM sodium EDTA, 0.5M NaCl, 0.05% Sodium Azide, 50 ug/ml Ampicillin, 5 mg/ml MgCl, 10 mg/ml BSA, 3 ul/ml Protease Inhibitor Cocktail solution (Sigma, P1860), 20 ug/ml goat anti-mouse IgG Fc-HRP conjugate.
Rapid Wash Solution: 50 mM Tris-HCl buffer, pH8, 0.5M NaCl, 0.3% Tween-20.
Blocking buffer (for regular immuno-detection): PBS with 1% BSA.
Buffer for regular immuno-detection: PBS buffer.
Antigen: Tat-flag expressed in CHO cells.
Primary antibody: mouse anti-Flag antibody.
2^{nd} Antibody: Goat anti-mouse whole IgG-HRP conjugate.
Substrate: Diaminobenzidine (DAB) (SIGMA's product, D4418)
Mayer's hematoxylin.
Glass slides.

### Sample Treatment:

3 day old transfected CHO/Tat-flag cells which could express and secrete engineered protein Tat-flag were harvested and cytospun onto slides (500,000 cells/slide), then were fixed with formalin and further treated with 3% hydrogen peroxide to inactivate the endogenous peroxidase.

### Repid Immuno-Detection:

1. Mixed 10 ul of primary antibodies with 1 ml (sufficient for 5 slides) of the one-step rapid reaction solution; directly overlay the pre-treated cells fixed on slide with 200 ul/slide of the primary antibody-supplemented one-step rapid reaction solution, and incubated for 30 minute at room temperature.
2. Rinsed slides 3 times with 2 ml the rapid wash solution and one time with 2 ml distilled water.
3. Developed color on the section with DAB solution for approximate 2 min until a desired color appears and then stopped the development by rinse slides with distilled water.
4. Incubated the sides in solution of Mayer's hermatoxyin for 3 min at room temperature for counterstain, then gently wash the slides with distilled water before micro-photography.

### Regular Immuno-Detection:

1. Incubated the slides with 200 ul/slide blocking buffer for 2 hr.
2. Wash the slides three times with 3 ml PBS.
3. Incubated the slides with 200 ul/slide primary antibody solution (1/100-diluted with PBS/1% BSA) for 2 hours at room temperature.
4. Wash the slides three times with 3 ml PBS.
5. Incubated the slides with 200 ul/slide 2nd antibody solution (1/100-diluted with PBS/1% BSA) for 1 hours at room temperature.
6. Wash the slides three times with 3 ml PBS.
7. Developed color on the section with DAB solution for approximate 2 min until a desired color appears and then stopped the development by rinse slides with distilled water.
8. Incubated the sides in solution of Mayer's hermatoxyin for 3 min at room temperature for counterstain, then gently wash the slides with distilled water before micro-photography.

### Results:

This experiment examined both the Universal Rapid Immuno-detection method and regular Immuno-detection method in testing the expression of a Flag-tagged HIV transcription factor protein Tat-flag in CHO cells by immunocytochemistry (ICC). The results by both methods are very similar but the testing time and procedure are great different: 30 minutes and 4 steps (by a rapid method) vs. 5 hours and 8 steps (by a regular method).

1-32. (canceled)

33. Sew) A Universal Rapid Immuno-detection method for detecting a specific protein, which consists of the steps of
1) immobilizing said specific protein on a solid phase;
2) combining, at one time, said specific protein immobilized on said solid phase, with a liquid phase comprising a Non-specific Competitor, a primary antibody, and a Specific Indicator, wherein said primary antibody binds directly to said specific protein and wherein said Specific Indicator comprises a protein that binds directly to said primary antibody on a portion of said primary antibody that does not interfere with binding of said primary antibody to said specific protein to form a specific protein-primary antibody Specific Indicator complex and wherein said primary antibody comprises an Fc-portion and said Specific Indicator is a protein recognizing the Fc-portion of said primary antibody;
3) separating said complex on said solid phase from said liquid phase; and
4) detecting a quantity of said specific protein.

34. The Universal Rapid Immuno-detection method of claim 33 wherein said liquid phase comprises a weight ratio of said Non-specific Competitor vs. said primary antibody plus said Specific Indicator of about 1,000 to about 100,000:1.

35. The Universal Rapid Immuno-detection method of claim 35 wherein the weight ratio of said Non-specific Competitor vs. said primary antibody plus Specific Indicator is about 5,000 to about 50,000:1.

36. The Universal Rapid Immuno-detection method of claim 33 wherein said Non-specific Competitor is selected from the group consisting of normal Igs, serum, albumin, Casein, Gelatine, chicken egg white, non-fat milk powder, and a mixture thereof

37. The Universal Rapid Immuno-detection method of claim 74 wherein said Specific Indicator is Protein L.

38. The Universal Rapid Immuno-detection method of claim 33 wherein said Specific Indicator is an Fc receptor protein.

39. The Universal Rapid Immuno-detection method of claim 41 wherein said Fc receptor protein is CD64.

40. The Universal Rapid Immuno-detection method of claim 72 wherein said capture protein is an antibody or a receptor protein.

41. The Universal Rapid Immuno-detection method of claim 33 wherein said liquid phase comprises a salt at a concentration of about 300 mM to about 1 M.

42. The Universal Rapid Immuno-detection method of claim 33 wherein said Immuno-detection is selected from the group consisting of Immuno-blot, ELISA, Immunohistochemistry, Immunocytochemistry, Flow Cytometry, and receptor-ligand binding assay.

43. The Universal Rapid Immuno-detection method of claim 33 wherein said solid phase is selected from the group consisting of Immuno-blotting membranes, ELISA plates, and Glass Slides.

44. The method of claim 33 wherein said liquid phase further comprises a Cofactor, a protease inhibitor or both.

45. A Universal Rapid Immuno-detection method for detecting a specific protein, which consists of the steps of
1) immobilizing a capture protein on a solid phase;
2) combining, at one time, said capture protein immobilized on said solid phase, with a liquid phase comprising said specific protein, a Non-specific Competitor, a primary antibody, and a Specific Indicator, wherein said capture protein binds directly to said specific protein, wherein said primary antibody binds directly to said specific protein and wherein said Specific Indicator comprises a protein that binds directly to said primary antibody on a portion of said primary antibody protein that does not interfere with binding of said primary antibody to said specific protein to form a capture protein-specific protein-primary antibody-Specific Indicator complex and wherein said primary antibody comprises an Fc-portion and said Specific Indicator is a protein recognizing the Fc-portion of said primary antibody and said Specific Indicator is a protein or antibody which does not bind to said capture protein;
3) separating said complex on said solid phase from said liquid phase; and
4) detecting a quantity of said specific protein.

46. A Universal Rapid Immuno-detection method for detecting a specific protein, which consists of the steps of
1) immobilizing said specific protein on a solid phase;
2) combining, at one time, said specific protein immobilized on said solid phase, with a liquid phase comprising a Non-specific Competitor, a primary antibody, and a Specific Indicator, wherein said primary antibody binds directly to said specific protein and wherein said Specific Indicator comprises a protein that binds directly to said primary antibody on a portion of said primary antibody that does not interfere with binding of said primary antibody to said specific protein to form a specific protein-primary antibody-Specific Indicator complex and wherein said primary antibody comprises a constant light chain region and said Specific Indicator is a protein recognizing the constant light chain region of said primary antibody;
3) separating said complex on said solid phase from said liquid phase; and
4) detecting a quantity of said specific protein.

47. A Universal Rapid Immuno-detection method for detecting a specific protein, which consists of the steps of
1) immobilizing a capture protein on a solid phase;
2) combining, at one time, said capture protein immobilized on said solid phase, with a liquid phase comprising said specific protein, a Non-specific Competitor, a primary antibody, and a Specific Indicator, wherein said capture protein binds directly to said specific protein, wherein said primary antibody binds directly to said specific protein and wherein said Specific Indicator comprises a protein that binds directly to said primary antibody on a portion of said primary antibody that does not interfere with binding of said primary antibody to said specific protein to form a capture protein-specific protein-primary antibody-Specific Indicator complex and wherein said primary antibody comprises a constant light chain region and said Specific Indicator is a protein recognizing the constant light chain region of said primary antibody and said Specific Indicator is a protein or antibody which does not bind to said capture protein;
3) separating said complex on said solid phase from said liquid phase; and
4) detecting a quantity of said specific protein.

48. The method of claim 33, wherein said specific protein immobilized on said solid phase, is combined with said liquid phase for about 30 minutes.

49. The method of claim 33 wherein said solid phase is separated from said liquid phase by washing said solid phase.

50. The Universal Rapid Immuno-detection method of claim 33 wherein said Specific Indicator is an antibody recognizing said Fc-portion of said primary antibody.

51. The Universal Rapid Immuno-detection method of claim 73 wherein said Specific Indicator is an antibody recognizing said Fc-portion of said primary antibody.
